(19) United States
(12) Patent Application Publication
(10) Pub. No.: US 2025/0275810 A1
(43) Pub. Date: Sep. 4, 2025

(54) SYSTEMS AND METHODS FOR NAVIGATING HIDDEN ANATOMIC PASSAGEWAYS

(71) Applicant: INTUITIVE SURGICAL OPERATIONS, INC., Sunnyvale, CA (US)

(72) Inventors: Serena H. Wong, Los Altos, CA (US); Samuel Raybin, San Jose, CA (US); Jennifer Golden, Norton, MA (US)

(21) Appl. No.: 19/064,448

(22) Filed: Feb. 26, 2025

Related U.S. Application Data
(60) Provisional application No. 63/559,730, filed on Feb. 29, 2024.

(51) Int. Cl. A61B 34/20; A61B 8/00; A61B 8/12; A61B 17/00; A61B 90/00

(52) U.S. Cl. CPC A61B 34/20; A61B 8/4416; A61B 8/445; A61B 17/00234; A61B 8/12; A61B 2017/0034; A61B 2034/2063; A61B 2034/2065; A61B 2090/367; A61B 2090/3784

(57) ABSTRACT

A system includes a flexible elongate device including an optical imaging device and a working channel, an acoustic imaging device carried by the flexible elongate device and configured to generate a plurality of acoustic images of a region of interest, and an interventional tool extendable through the working channel of the flexible elongate device. The system also includes a control system configured to receive the plurality of acoustic images of the region of interest from the acoustic imaging device and provide guidance for inserting the interventional tool along a path through an anatomic orifice and an anatomic passageway of the region of interest, wherein the guidance is generated based on the plurality of acoustic images.

### CROSS-REFERENCED APPLICATIONS

This application claims priority to and benefit of U.S. Provisional Application No. 63/559,730 filed Feb. 29, 2024 and entitled “Systems and Methods for Navigating Hidden Anatomic Passageways,” which is incorporated by reference herein in its entirety.

### FIELD

The present disclosure relates to navigation of anatomic passageways, and more particularly to identifying and navigating anatomic passageways that are not directly visible with an optical imaging device.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert a minimally invasive medical instrument (including surgical, diagnostic, therapeutic, and/or biopsy instruments) to reach a target tissue location. One such minimally invasive technique is to use a flexible elongate device which can be inserted into anatomic passageways and navigated toward a region of interest within the patient anatomy. An interventional tool may extend from the flexible elongate device to perform an interventional procedure at the region of interest. If the region of interest is not directly visible with an optical imaging device of the flexible elongate device, deployment of the interventional tool may unintentionally contact sensitive surrounding tissue. Systems and methods for identifying and navigating anatomic passageways that may not be directly visualized with an optical imaging device.

### SUMMARY

The following presents a simplified summary of various examples described herein and is not intended to identify key or critical elements or to delineate the scope of the claims.

Consistent with some examples, a system may comprise a flexible elongate device including an optical imaging device and a working channel, an acoustic imaging device carried by the flexible elongate device, the acoustic imaging device configured to generate a plurality of acoustic images of a region of interest, an interventional tool extendable through the working channel of the flexible elongate device, and a control system configured to: receive the plurality of acoustic images of the region of interest from the acoustic imaging device and provide guidance for inserting the interventional tool along a path through an anatomic orifice and an anatomic passageway of the region of interest, wherein the guidance is generated based on the plurality of acoustic images.

Consistent with some examples, a method may comprise receiving, by a control system, a plurality of acoustic images of a region of interest, the plurality of acoustic images generated by an acoustic imaging device carried by a flexible elongate device including an optical imaging device and a working channel, and providing, by the control system, guidance for inserting an interventional tool along a path through an anatomic orifice and an anatomic passageway of the region of interest, the interventional tool extendable through the working channel of the flexible elongate device, wherein the guidance is generated based on the plurality of acoustic images.

Consistent with some examples, a non-transitory machine-readable medium may comprise a plurality of machine-readable instructions which when executed by one or more processors associated with a device are adapted to cause the one or more processors to: receive a plurality of acoustic images of a region of interest, the plurality of acoustic images generated by an acoustic imaging device carried by a flexible elongate device including an optical imaging device and a working channel, and provide guidance for inserting an interventional tool along a path through an anatomic orifice and an anatomic passageway of the region of interest, the interventional tool extendable through the working channel of the flexible elongate device, wherein the guidance is generated based on the plurality of acoustic images.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 illustrates an instrument system extended within an anatomic structure, according to some examples.

FIG. 2 illustrates an instrument system near a region of interest, according to some examples.

FIG. 3 illustrates a method for identifying and navigating anatomic passageways hidden from endoscopic view, according to some examples.

FIG. 4 illustrates an instrument system including a flexible elongate device and a separable acoustic imaging tool, according to some examples.

FIG. 5 illustrates a method for identifying and navigating anatomic passageways hidden from endoscopic view, according to some examples.

FIG. 6 illustrates guidance for inserting an interventional tool along a path, according to some examples.

FIG. 7 illustrates guidance for inserting an interventional tool along a path, according to some examples.

FIG. 8 illustrates an instrument system including a flexible elongate device and a separable acoustic imaging tool, according to some examples.

FIG. 9 illustrates an instrument system including a flexible elongate device and an integrated acoustic imaging tool, according to some examples.

FIG. 10 illustrates a method for identifying and navigating anatomic passageways hidden from endoscopic view, according to some examples.

FIG. 11 illustrates guidance for inserting an interventional tool along a path, according to some examples.

FIG. 12 illustrates a method for identifying and navigating anatomic passageways hidden from endoscopic view, according to some examples.

FIG. 13 illustrates guidance for inserting an interventional tool along a path, according to some examples.

FIG. 14 illustrates a simplified diagram of a medical system, according to some examples.

FIG. 15A illustrates a simplified diagram of a medical instrument system, according to some examples.

FIG. 15B illustrates a simplified diagram of a medical instrument including a medical tool within a flexible elongate device, according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

Some medical procedures involve accessing anatomic passageways that may be difficult to identify and navigate due to their size, complexity, and/or location. For example, an endoscopic retrograde cholangiopancreatography (ERCP) may involve navigation within the pancreaticobiliary junction. The common bile and pancreatic ducts may be difficult to identify and navigate because they are small, because they are not consistent from patient to patient, and/or because they are located behind the sphincter of oddi and the ampulla of vater and thus not able to be directly visualized by an optical imaging device stationed in the duodenum. The systems described herein may include a flexible elongate device such as an endoscope or a catheter fitted with the optical imaging device. Due to its size, stiffness, and/or articulation limits, the flexible elongate device may be unable to access some anatomic passageways to regions of interest. Instead, the flexible elongate device may serve as a deployment stage for an interventional tool to access the region of interest. If the region of interest is not directly visible with an optical imaging device of the flexible elongate device, deployment of the interventional tool from the flexible elongate device may unintentionally result in contact with sensitive surrounding tissue. For example, in an ERCP procedure, a catheter (e.g., an interventional tool) may extend from an endoscope (e.g., a flexible elongate device) and into the bile and pancreatic ducts. If the catheter is not accurately oriented, advancement of the catheter may cause tissue trauma, and repeated attempts at insertion may result in inflammation, potentially leading to pancreatitis. The flexible elongate devices described herein may carry an acoustic imaging device, such as an ultrasound imaging device, with an associated localization device. The resulting localized acoustic imaging data may be used to identify a path to the region of interest that is not directly visible by the optical imaging device of the flexible elongate device. The interventional tool deployed from the flexible elongate device may follow the identified path to the region of interest while avoiding sensitive anatomic structures in the vicinity of the path.

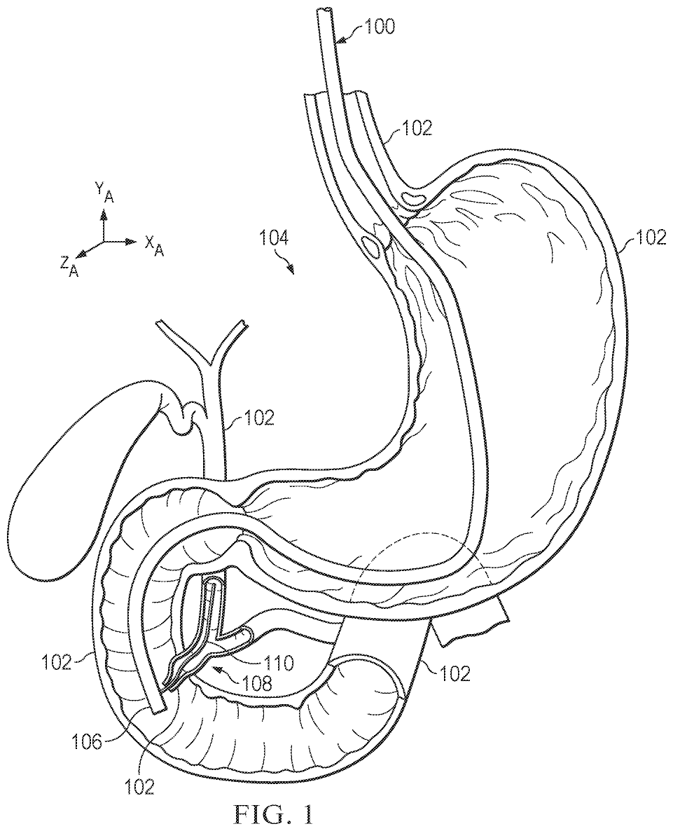

FIG. 1 illustrates a flexible elongate device 100 extended within anatomic passageways 102 of an anatomic structure 104. In some examples the anatomic structure 104 may be a gastrointestinal tract and the anatomic passageways 102 may include the esophagus, the stomach, the duodenum, the ampulla of vater, the pancreatic duct, and the common bile duct, among other anatomic passageways. The anatomic structure 104 has an anatomic frame of reference (X_{A}, Y_{A}, Z_{A}). The flexible elongate device 100 may include, for example, a catheter or an endoscopic device. An endoscopic device may include a specialized endoscopic device such as a bronchoscope or a duodenoscope. In some examples, the flexible elongate device 100 includes multiple elongate devices, for example, a system of elongate devices. The system of elongate devices may include at least one endoscope disposed within an endoscope. In one example, the system of elongate devices may include a forward viewing endoscope within a duodenoscope, among other examples. A distal end portion 106 of the flexible elongate device (or group of elongate devices) 100 may be advanced into an anatomic opening (e.g., a patient mouth) and through the anatomic passageways 102. The flexible elongate device 100 may provide a platform from which to deploy an interventional tool 110 to perform a medical procedure at or near a region of interest 108. The flexible elongate device 100 may be suitable for use in, for example, surgical, exploratory, diagnostic, therapeutic, or any other type of medical procedure. In some examples, the flexible elongate device 100 may be used for ductal cannulation, ERCP, biopsy, ablation, or other types of procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose robot-assisted medical systems.

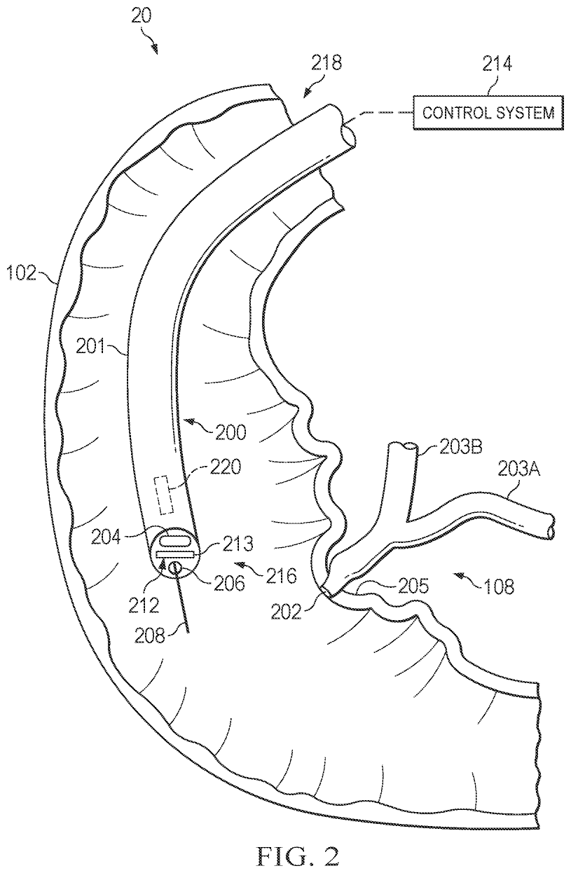

FIG. 2 illustrates a system 20 including a flexible elongate device 200 (e.g., the flexible elongate device 100) in an anatomic passageway 102, near the region of interest 108. The flexible elongate device 200 may include a flexible body 201 and an optical imaging device 204 extending within the flexible elongate body. The flexible elongate device 200, in some examples, is supported by or guided through a working channel of a duodenoscope. The optical imaging device 204 may include, for example, a monoscopic or stereoscopic camera. In some examples, the optical imaging device 204 may be the optical imaging device of an endoscopic device such as a bronchoscope or a duodenoscope. In some examples, the optical imaging device 204 may include a light source to provide illumination of anatomic tissues and structures surrounding the optical imaging device 204. The optical imaging device 204 may be integral with or removable from the flexible body 201. The flexible elongate device 200 may also include a working channel 206 extending through the flexible body 201. The working channel 206 may extend from a proximal end 218 to a distal end 216 of the flexible elongate device 200. Any of a variety of tools, instruments, devices, and/or fluids may be introduced or removed from the patient anatomy through the working channel 206 of the flexible elongate device 200.

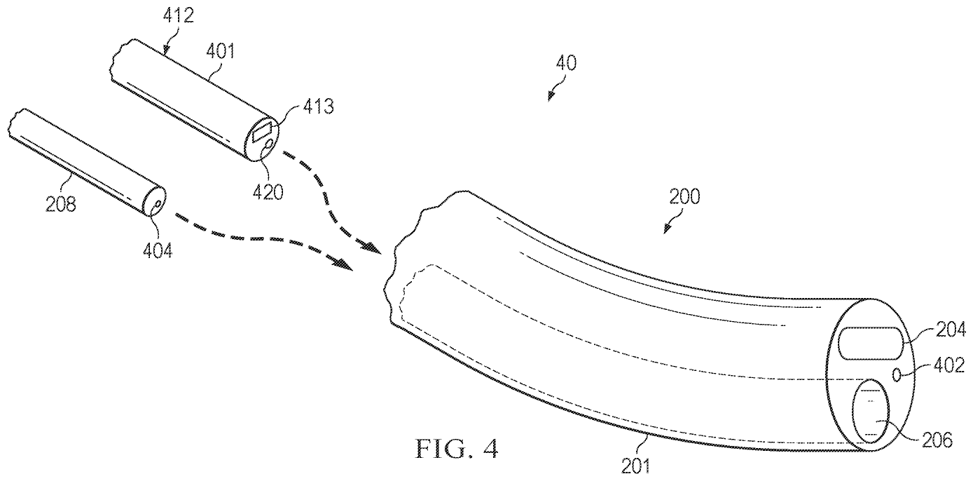

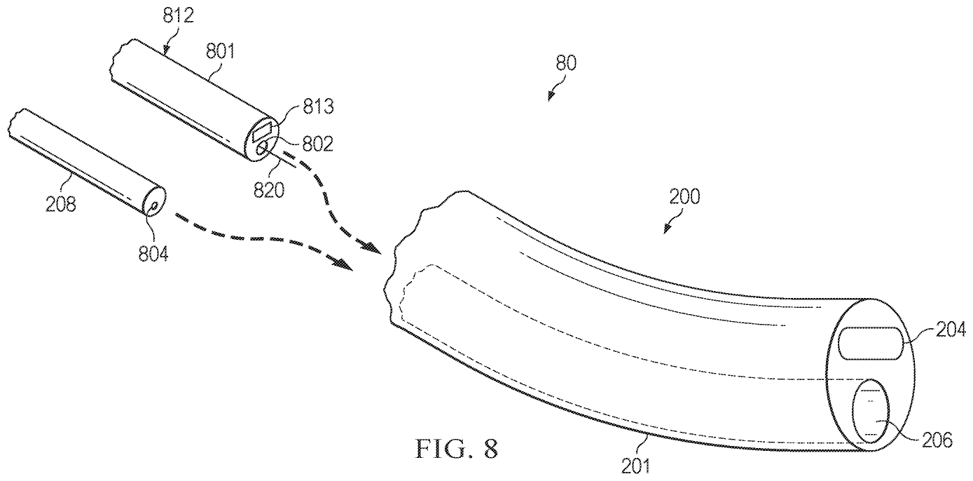

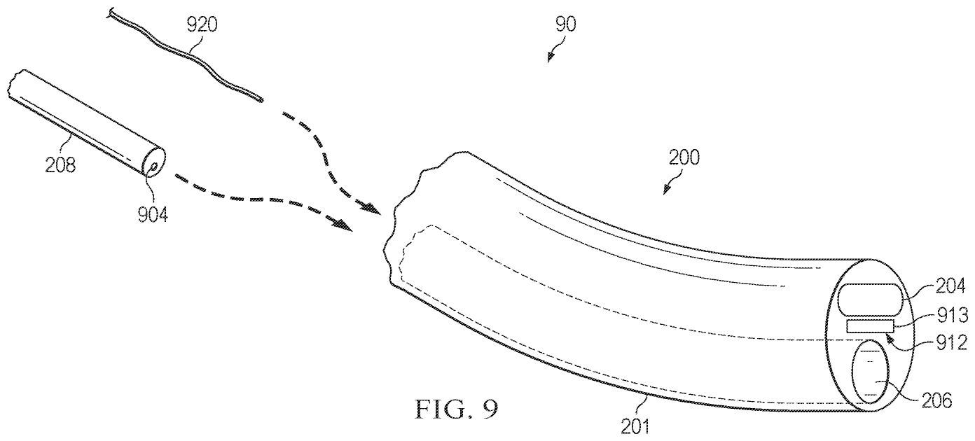

The system 20 may also include an acoustic imaging device 212 carried by the flexible elongate device 200. The acoustic imaging device 212 may include, for example, one or more sonic transducers 213, such as ultrasonic transducers, that may generate acoustic imaging data used to produce acoustic images, such as ultrasound images. In some examples (e.g. FIG. 9), components of the acoustic imaging device 212, such as the transducers, may be integral with the flexible body 201. In other examples (e.g., FIGS. 4 and 8), the acoustic imaging device 212 system or components thereof may be included in an acoustic imaging tool that is removable or separable from the flexible body 201 of flexible elongate device 200. The separable acoustic imaging tool may be extendable within the working channel 206 or within a dedicated imaging channel of the flexible body 201.

The system 20 may also include an interventional tool 208 (e.g., interventional tool 110) which may be extendable through the working channel 206 and distally of the distal end 216 of the flexible elongate device 200. The interventional tool 208 may be selected to perform any of a variety of functions or procedures. For example, the interventional tool 208 may include a catheter, a treatment tool, a biopsy tool, a sensor tool, or any other type of tool used in a medical intervention.

Optionally, the system 20 may include a localization device 220 that may be used to reference the acoustic image data from the acoustic imaging device 212 to the region of interest 108. In some examples, the localization device 220 may include a position, orientation, and/or shape sensor that has a known spatial relationship to the location device 220 and may generate localization data used to localize the acoustic imaging data to the frame of reference (e.g., the X_{A}, Y_{A}, Z_{A} frame of reference) of the region of interest 108. The localized acoustic imaging data may then provide guidance for introducing the interventional tool 208 within the region of interest. In other examples, the localization device 220 may include a guide device, such as a guidewire, that may be visualized in the acoustic images as it extends into the region of interest. The guide device may provide guidance for introducing the interventional tool 208 within the region of interest.

The system 20 may also include a control system 214. In some examples, the control system 20 may be a component of or operate in coordination with a control system of a robot-assisted medical system (e.g. the control system 1412). The control system 214 may be used to control operation of any of the functions of the flexible elongate device 200, including processing image data from the optical imaging device 204 and the acoustic imaging device 212, controlling or processing information from the localization device, and/or controlling motion of the flexible elongate and/or the interventional tool.

In some examples, the flexible elongate device 200 may be positioned near an anatomic orifice 202 (e.g., an anatomic sphincter or duct) to provide a platform for introducing the interventional tool 208 along a path through the anatomic orifice 202 and into one or more anatomic passageways 203A, 203B in the region of interest 108. The optical imaging device 204 may be used to navigate the flexible elongate device 200 within the anatomic passageways 102 and position the flexible elongate device 200 near the anatomic orifice 202. The region of interest 108, including the anatomic passageways 203A, 203B, may be hidden or otherwise obscured from the field of view of the optical imaging device 204 of the flexible elongate device 200 by the wall 205 of the anatomic passageway 102. The acoustic imaging device 212 may be positioned near or in contact with the anatomic orifice 202 to generate a plurality of acoustic images of the region of interest 108. The acoustic images may help to visualize and identify a path through the anatomic orifice 202 and passageways 203A, 203B hidden to the optical imaging device 204. For some procedures, the acoustic images may also help to visualize a target tissue such as a tumor or lesion in the region of interest, and the identified path may further lead to the target. In some examples, the path may be identified by an operator viewing one or more of the displayed acoustic images. In some examples, the path may be identified using graphical analysis techniques or other detection methods performed by the control system 214. In some examples, the path may be identified using a combination of operator and control system evaluations. The paths described herein may be navigational paths which help to insert an interventional tool down the appropriate hidden passageway, preventing undesirable consequences that may arise from traversing unintended passageways. In some examples, the path is an optimized route, decreasing the amount of contact that an interventional tool makes with tissue and decreasing the time required to perform the surgery. Guidance for inserting the interventional tool along the path may be provided, for example, by displaying the acoustic images including the path or displaying a model generated by the acoustic images that includes the path.

An example of a procedure that may be performed with the flexible elongate device 200 is a ductal cannulation in which the interventional tool may be a cannula that is inserted into the sphincter of oddi (e.g., orifice 202) and navigates into the pancreatic duct (e.g., passageway 203A) or the bile duct (e.g., passageway 203b) to perform any of a variety of procedures including stone removal, biopsy, dilation, stenting, or ablation. In some examples, the flexible elongate device 200 may be used to create a fistula to pass through the wall of the duodenum directly to the cystic duct, bypassing the junction pancreatic duct.

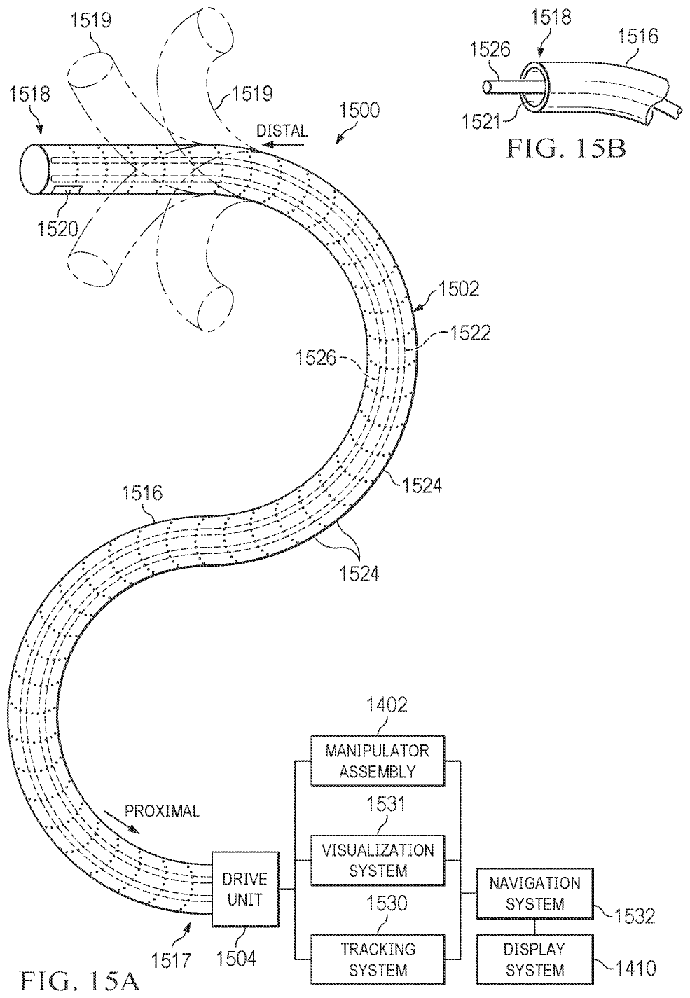

Although the optical imaging device 204, the opening of the working channel 206, the interventional tool 208, and the acoustic imaging device 212 are shown disposed at the distal end 216 of the flexible elongate device 200, it is understood that some or all of these features can be side-facing, for example, directed out a side the flexible elongate device 200, depending on the particular use case. In some examples, the flexible elongate device 200 may be a component of a robotically assisted medical instrument system or a manually-controlled medical instrument system that controls articulation and insertion/retraction of the flexible elongate device 200. An example of a medical instrument system including a flexible elongate device that is bendable and steerable in multiple degrees of freedom is described below in FIGS. 15A and 15B (e.g., system 1500).

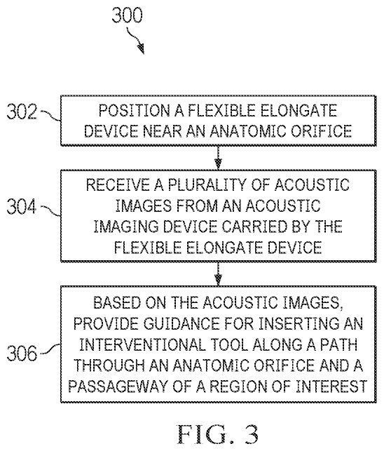

FIG. 3 is a flowchart illustrating a method 300 for providing guidance for introducing an interventional tool into a region of interest. The system 20 described with respect to FIG. 2 may be used in performing the method 300. At an optional process 302, a flexible elongate device may be positioned near an anatomic orifice. For example, the flexible elongate device 200 may be positioned near the anatomic orifice 202. In some examples, the flexible elongate device 200 may be manually inserted and steered toward the anatomic orifice 202. In other examples, the flexible elongate device 200 may be inserted and steered with robot-assistance. The optical imaging device 204 may be used to guide navigation through the anatomic passageways 102 and to visualize the anatomic orifice 202. If the optical imaging device 204 is positioned on the distal end 216 of the flexible elongate device 200, insertion of the flexible elongate device 200 may stop short of the anatomic orifice 202 so that the anatomic orifice 202 remains in view of the optical imaging device 204. If the optical imaging device 204 is side-facing or extends out a working channel in the side of the flexible elongate device 200, the flexible elongate device 200 may be navigated slightly beyond the anatomic orifice 202 to position the optical imaging device 204 appropriately. In other examples, where the optical imaging device 204 is integrated with the flexible elongate device 200, the flexible elongate device 200 may bend such that the distal end 216 of the flexible elongate device 200 is directed at the anatomic orifice 202. Ultimately, a wide variety of positioning strategies are contemplated, as the positioning of the flexible elongate device 200 near the anatomic orifice 202 may depend on the location of the optical imaging device 204 with respect to the acoustic imaging device 212. For example, the flexible elongate device 200 may be navigated beyond, in line with, or short of the anatomic orifice 202 depending on the spatial relationship between the optical imaging device 204 and the acoustic imaging device 212. Positioning the flexible elongate device 200 near or in contact with the anatomic orifice 202 may prepare the acoustic imaging device 212 to obtain a plurality of acoustic images of the region of interest 108. In some examples, the acoustic imaging device extends from the working channel 206 to contact with the anatomic orifice 202.

At a process 304, a plurality of acoustic images, such as ultrasound images, may be received from an acoustic imaging device carried by the flexible elongated device. For example, a plurality of acoustic images may be received from the acoustic imaging device 212 carried by the flexible elongate device 200. After the acoustic imaging device 212 is positioned near or in contact with the anatomic orifice 202 at the process 302, the acoustic imaging device 212 may be moved to different orientations and positions near the anatomic orifice 202 to obtain the plurality of acoustic images from different configurations. Such a maneuver of the flexible elongate device 200 may include twisting, rotating, and/or dragging the distal end 216 with respect to the anatomic orifice 202. Moving the acoustic imaging device 212 to various configurations may allow the control system to gather sufficient image data for use in identifying the hidden anatomic passageway and providing a guidance path within the region of interest. The plurality of images may be transmitted to the control system 214.

At a process 306, one or more of the plurality of acoustic images may be used to provide guidance for inserting an interventional tool along a path through an anatomic orifice and a passageway of a region of interest. For example, the acoustic image data or the acoustic images from the acoustic imaging device 212 may be used to provide guidance for inserting the interventional tool 208 along a path through the anatomic orifice 202 and the passageway 203A of the region of interest 108. In some examples, providing the guidance may include generating and displaying a three-dimensional model of the region of interest 108 generated from the plurality of acoustic images. The anatomic passageways visible in the model may be used to identify a path within the region of interest for inserting the interventional tool. In other examples, providing the guidance may include displaying one or more of real-time acoustic images that include a passageway within the region of interest as the interventional tool or a guide device, such as a guidewire over which the interventional tool may be inserted, is inserted along a path through the passageway in the real-time acoustic image. Optionally, the process of providing guidance may include identifying the path through the anatomic orifice 202 and the hidden passageways 203A. The path can be identified by a clinician, by the control system, or by a combination of the two. Generally, the path may be a navigational path, a guidance path, or any other type of path. The identified paths may be used to accurately and precisely insert the interventional tool 208 into otherwise hidden anatomic passageways while minimizing or eliminating tissue trauma. The guidance may assist an operator in aligning the flexible elongate device 200 with the identified path so that the interventional tool 208 can be inserted into the correct hidden anatomic passageway 203A, 203B. In some examples, the guidance may include visual, audio, haptic, or any other type of guidance. In some examples, the guidance may include insertion instructions or information identifying the names and locations of passageways that are hidden. In other examples, the guidance is visual display of the identified and marked path on a 2D image, 3D image, an intra-operative 3D model, or a pre-operative 3D model.

After or while the guidance is provided, the method 300 may, optionally, include inserting the interventional instrument along the path. For example, the interventional tool 208 may be inserted along an identified path through the orifice 202 and the passageway 203A using the guidance generated from the acoustic images.

FIG. 4 illustrates a system 40 which may be similar to the system 20, with differences as described. In this example an acoustic imaging device 412 (e.g. the acoustic imaging device 212) is a separable tool from the body 201 of the flexible elongate device 200. The separable acoustic imaging device 412 may be extendable through the working channel 206 of the flexible body 201 or through any channel of the body 201. In this example, the acoustic imaging device 412 includes an elongate flexible body 401 that includes one or more sonic transducers 413, such as ultrasound transducers, and a localization device 420 (e.g. the localization device 220). In this example, the localization device 420 may include an localization sensor such as an electromagnetic (EM) position sensor, an optical fiber shape sensor, or any other type of position or shape sensor. A localization sensor 402 may be housed in the body 201 of the flexible elongate device 200 and may be fixed relative to the acoustic imaging device 412. The localization device 420 and thus the acoustic imaging device 412 may be registered to a common reference frame as the flexible elongate device 200 and the localization sensor 402 so that the location of the acoustic imaging device 412 may be known relative to the flexible elongate device 200. As the acoustic imaging device 412 gathers image data, the location data for the sonic transducers 413 may be tracked by the localization sensor 420 used in building a three-dimensional model of the region of interest. Optionally, the model may be segmented to identify tissue and passageways. Further, the location data may be used in identifying a path through the anatomic passageways in the model and, optionally, to a target. The location data may also be used to align the flexible elongate device 200 with the path before insertion of the interventional tool 208. In some examples, the localization device 420 is not needed. Rather, the location of the acoustic imaging device 412 with respect to the flexible elongate device 200 may be tracked or registered using keying techniques and/or with reference to the insertional distance of the acoustic imaging device 412 and the flexible elongate device 200.

The working channel 206 may be sized to receive the acoustic imaging device 412 or the interventional tool 208. Accordingly, the acoustic imaging device 412 may be removed from the working channel 206 after the plurality of acoustic images are captured and the interventional tool 208 may be extended through the working channel 206 to be inserted through the anatomic orifice 202 and along the identified path to navigate the hidden anatomic passageways 203A, 203B. In some examples, the interventional tool 208 may be steerable and may include a localization sensor 404 for determining its shape and location. In some examples, the localization sensor 404 is not needed. Rather, the location of the interventional tool 208 with respect to the flexible elongate device 200 may be tracked or registered using keying techniques and/or with reference to the insertional distance of the interventional tool 208 and the flexible elongate device 200.

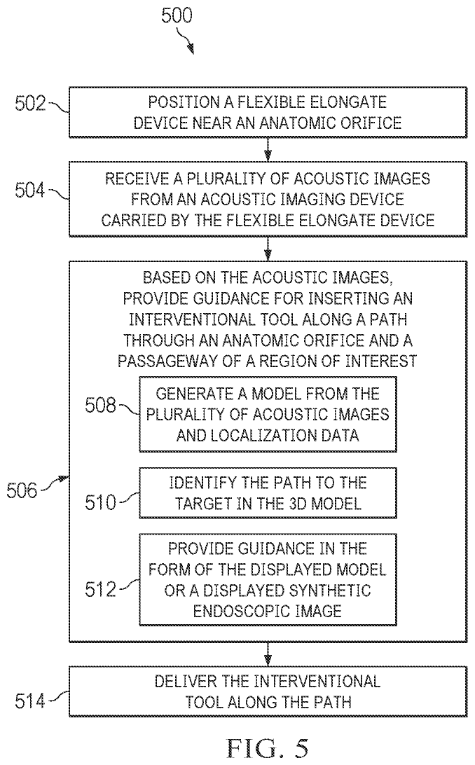

FIG. 5 is a flowchart illustrating a method 500 of providing guidance for introducing an interventional tool into a region of interest. The system 40 described in FIG. 4 may be used in performing the method 500.

At an optional process 502 that may be similar to process 302, a flexible elongate device may be positioned near an anatomic orifice. For example, the flexible elongate device 200 may be positioned near the anatomic orifice 202. In some examples, the flexible elongate device 200 may be manually inserted and steered toward the anatomic orifice 202. In other examples, the flexible elongate device 200 may inserted and steered with robot-assistance. The optical imaging device 204 may be used to guide navigation through the anatomic passageways 102 and to visualize the anatomic orifice 202.

At a process 504, a plurality of acoustic images, such as ultrasound images, may be received from an acoustic imaging device carried by the flexible elongated device. For example, a plurality of acoustic images may be received from the acoustic imaging device 412 carried by the flexible elongate device 200. More specifically, the separable acoustic imaging device 412 may be received within the working channel 206 of the flexible elongate device 200. In some examples, the distal end of the acoustic imaging device 412 may be extended distally of the flexible elongate device 200 and may be articulated, rotated, or otherwise moved while obtaining a plurality of acoustic images from different positions and orientations. The acoustic imaging device 412 may be registered to the flexible elongate device 200 using the localization sensor 420 in conjunction with the localization sensor 402 on the flexible elongate device 200. In some examples, the location of the acoustic imaging device 412 with respect to the flexible elongate device 200 is tracked or registered using keying techniques and/or with reference to the insertional distance of the acoustic imaging device 412 and the flexible elongate device 200. The plurality of images may be transmitted to the control system 214.

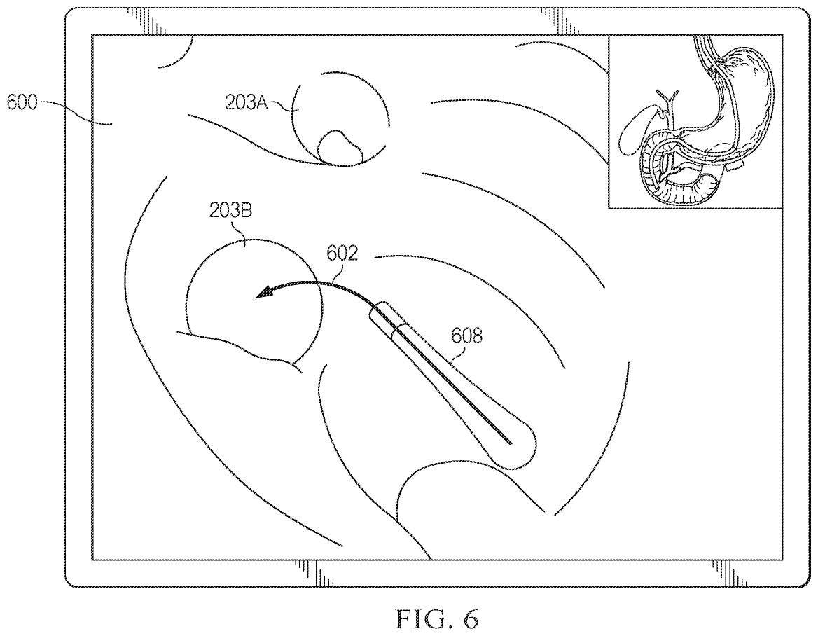

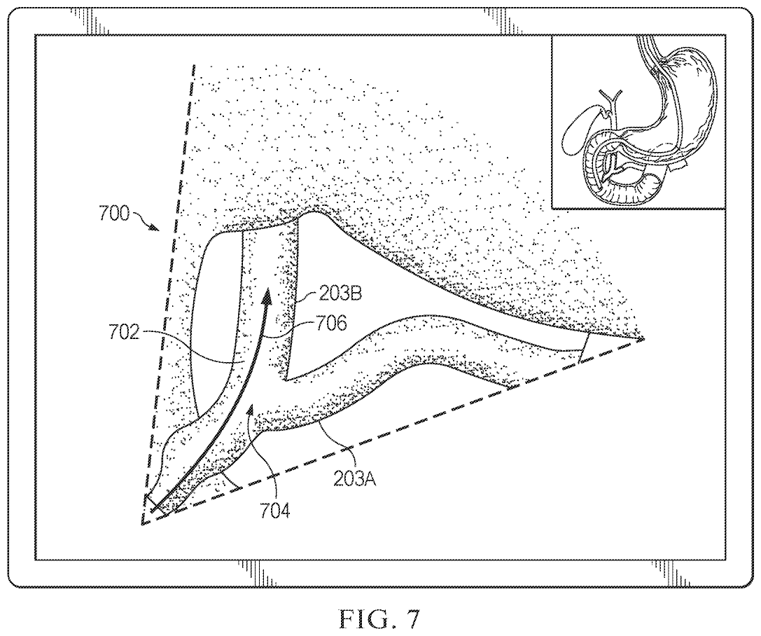

At a process 506, one or more of the plurality of acoustic images may be used to provide guidance for inserting an interventional tool along a path through an anatomic orifice and a passageway of a region of interest. For example, the acoustic image data or the acoustic images from the acoustic imaging device 412 may be used to provide guidance for inserting the interventional tool 208 along a path through the anatomic orifice 202 and the passageway 203A of the region of interest 108. In this example providing the guidance may include a process 508 of generating a model, such as a three-dimensional model, of the region of interest 108 from the plurality of acoustic images and the localization data from the localization sensor 420. At a process 510, the anatomic passageways visible in the model may be used to identify a path within the region of interest for inserting the interventional tool. For example, the path may extend through the anatomic orifice 202 and the hidden passageways 203A. The path can be identified by a clinician, by the control system, or by a combination of the two. In some examples, the path may be of a straight trajectory from the anatomic orifice 202 for traversal by a generally rigid interventional tool 208. In other examples, the path may be a curved path from the anatomic orifice 202 for traversal by a sensored, steerable interventional tool 208. At a process 512, the guidance may be provided in the form of a synthetic endoscopic image (FIG. 6) generated from the three-dimensional model or the displayed three-dimensional model (FIG. 7). A marker of the path may be displayed with the model or synthetic endoscopic image.

As shown in FIG. 6, guidance in the form of a synthetic endoscopic image 600 may be shown from a perspective in the region of interest 108 beyond the anatomic orifice 202, allowing for visualization of the hidden passageways 203A, 203B. The synthetic endoscopic image 600 may include a representation 608 of the interventional tool 208 based on the current predicted trajectory. The synthetic endoscopic image 600 may also include a path 602 identified in the process 510. Both may be used for navigation by the interventional tool 208 through the hidden anatomic passageways 203A, 203B. In other examples, a synthetic endoscopic image may be shown from the perspective of the optical imaging device 204 needed to align the flexible elongate device to deploy the interventional tool along a trajectory following the path. In such an example, the synthetic endoscopic image may display the trajectory of the identified path entering the anatomic orifice 202. In such an example, a real-time optical image may be provided on the display next to the synthetic endoscopic image, allowing for side-by-side comparison. The position and orientation of the flexible elongate device 200 may be adjusted manually or by the control system 214 until the real-time endoscopic image matches the synthetic endoscopic image. This matching and alignment process prepares the interventional tool 208 for deployment along the identified path.

As shown in FIG. 7, the guidance may include display of a 3D model 700 generated from the plurality of acoustic images. Because the 3D model 700 is registered to the flexible elongate device 200 and the steerable interventional tool 208, the position of the steerable interventional tool 208 is known with respect to the 3D model 700. Accordingly, a path 702 identified in the process 510 may be displayed from the 3D model 700. The localization sensor 404 on the steerable interventional tool 208 may provide a current location 704 of the interventional tool 208 and the current location 704 may be updated and shown in the display of the 3D model 700 as the navigational tool 208 follows the path 702. Optionally, a path marker 706 may be overlayed on the displayed model 700. In this way, the steerable interventional tool 208 can be maneuvered through the hidden passageways 203A, 203B, using the path 702 (and/or the marker 706 as guidance.

After or while the guidance is provided, the method 500 may, optionally, include a process 514 of inserting the interventional instrument along the path. For example, the acoustic imaging device 412 may be removed from the working channel 206, the interventional tool 208 may be inserted along an identified path through the orifice 202 and the passageway 203A using the guidance generated from the acoustic images.

FIG. 8 illustrates a system 80 which may be similar to the system 20, with differences as described. In this example an acoustic imaging device 812 (e.g. the acoustic imaging device 212) is separable tool from the body 201 of the flexible elongate device 200. The separable acoustic imaging device 412 may be extendable through the working channel 206 of the flexible body 201 or through any channel of the body 201. In this example, the acoustic imaging device 812 includes an elongate flexible body 801 that includes one or more sonic transducers 413, such as ultrasonic transducers, and a localization device 820 (e.g. the localization device 220) extendable through a guide channel 802 of the flexible body 801. In this example, the localization device 820 may include a guide device such as a guidewire or other structure that may follow a path within the region of interest. In some examples, identifying the path may include selecting a guidance image of the plurality of acoustic images that includes the path through the anatomic orifice 202 and the selected passageway 203A, 203B and optionally, to a target. The guidance image may be selected by an operator or by the control system based, for example on image analysis. A control system may, for example, employ artificial intelligence to compare the plurality of acoustic images to prior captured images having a path.

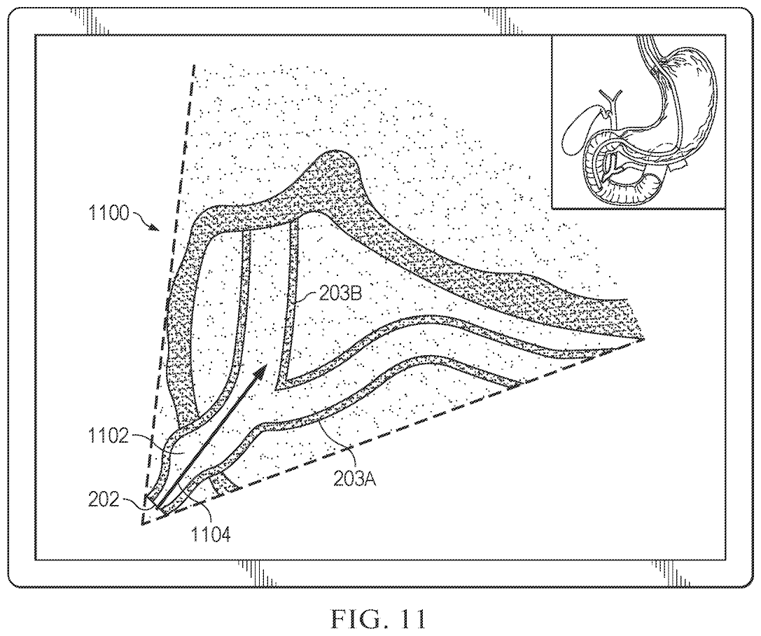

Using the selected guidance image (e.g., guidance image 1100 shown in FIG. 11), the system 80 may provide guidance for inserting the guidewire along the path. In some examples, the guidance is a display of the selected guidance image 1100. The guidance may further include a marker of the identified path (e.g. path 1002) to the target superimposed on the selected guidance image 1100. Alternatively, the path may simply be the visible passageways in the selected guidance image. To prepare for insertion of the interventional tool 208, the guide channel 802 housing the guidewire 820 may be aligned with the path visible in the selected guidance image 1100. The guidewire 820 is then inserted from the guide channel 802 that is aligned with the path. The location and movement of the guidewire may be visible in the selected guidance image as the guidewire is advanced along the path.

After the guidewire 820 has been inserted along the path, the interventional tool 208 can be delivered along the path. In some examples, delivering the interventional tool 208 includes removing the acoustic imaging device 812 from the working channel 206 while the guidewire 820 remains in place extended along the path. Once the acoustic imaging device 812 is removed, the interventional tool 208 may be inserted over the aligned guidewire 820 to deliver the interventional tool 208 along the path in the region of interest. In some examples, the interventional tool 208 may include a guide channel 804 which guides the interventional tool 208 over the guidewire 820.

FIG. 9 illustrates a system 90 (e.g., the system 20) that may be similar to the system 80 but wherein, unlike the system 80, an acoustic imaging device 912 including sonic transducers 91 is integrated with the body 201 of the flexible elongate device 200. A localization device 920 is a guide device such as a guidewire extendable through the working channel 206 of flexible elongate device 200. In this example, the working channel 206 serves as a guide channel for the guidewire 920. After the working channel 206 is aligned with the identified path through the region of interest, the guidewire 920 is inserted from the working channel 206 along the path, as previously described. The guidewire 920, as the localization device, serves as a placeholder, until the interventional tool 208 is inserted through the working channel 206 over the guidewire 920. In some examples, the interventional tool 208 may have a guide channel 904 which guides the interventional tool 208 over the guidewire 920.

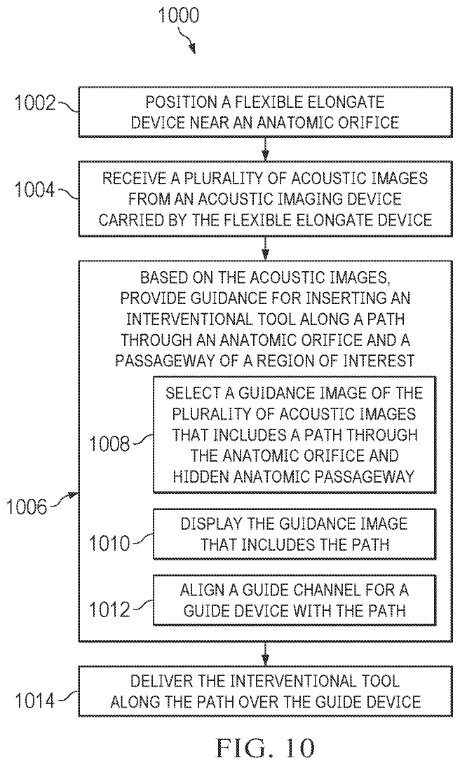

FIG. 10 is a flowchart illustrating a method 1000 of providing guidance for introducing an interventional tool into a region of interest. The systems 80 or 90 may be used in performing the method 1000.

At an optional process 1002 that may be similar to process 302, a flexible elongate device may be positioned near an anatomic orifice. For example, the flexible elongate device 200 may be positioned near the anatomic orifice 202. In some examples, the flexible elongate device 200 may be manually inserted and steered toward the anatomic orifice 202. In other examples, the flexible elongate device 200 may be inserted and steered with robot-assistance. The optical imaging device 204 may be used to guide navigation through the anatomic passageways 102 and to visualize the anatomic orifice 202.

At a process 1004, a plurality of acoustic images, such as ultrasound images, may be received from an acoustic imaging device carried by the flexible elongated device. For example, a plurality of acoustic images may be received from the acoustic imaging device 812, 912 carried by the flexible elongate device 200. In some examples, the separable acoustic imaging device 812 may be received within the working channel 206 of the flexible elongate device 200. The distal end of the acoustic imaging device 812 may be extended distally of the flexible elongate device 200 and may be articulated, rotated, or otherwise moved while obtaining a plurality of acoustic images from different positions and orientations. In some examples, with the acoustic imaging device 912 is integrated with the body 201 of the flexible elongate device 200, the flexible elongate device may be articulated, rotated, or otherwise moved while obtaining a plurality of acoustic images from different positions and orientations. The plurality of images may be transmitted to the control system 214.

At a process 1006, one or more of the plurality of acoustic images may be used to provide guidance for inserting an interventional tool along a path through an anatomic orifice and a passageway of a region of interest. For example, the acoustic image data or the acoustic images from the acoustic imaging device 412 may be used to provide guidance for inserting the interventional tool 208 along a path through the anatomic orifice 202 and the passageway 203A of the region of interest 108. In this example, providing the guidance may include a process 1008 of selecting a guidance image of the plurality of acoustic images. The guidance image may include a path through the anatomic orifice 202 and one of the anatomic passageways 203A, 203B. The guidance image may be selected by an operator or by the control system based, for example on image analysis.

At a process 1010, the guidance image including the path may be displayed. For example, a guidance image (e.g. guidance image 1100) including a path through the orifice 202 and the passageway 203A may be displayed on a display system (e.g. display system 1410). Optionally, a marker indicating the path may be overlayed on the guidance image. As shown in FIG. 11, a guidance image 1100 selected from the acoustic images may be displayed. In the guidance image 1100, the path 1102 from the orifice 202 through the passageways 203A,203B is visible. Optionally, a marker 1104, such as an arrow or line, may be overlayed on the guidance image to emphasize the trajectory of the path 1102.

At a process 1012, a guide channel for a guide device may be aligned with the path. For example, the guide channel 802 or the working channel 206 guiding the guidewire 920 may be aligned with the path 1102 and/or the marker 1104 visible in the selected guidance image 1100. The guidewire 820, 920 may then be inserted from the guide channel 802 that is aligned with the path. The location and movement of the guidewire may be visible in the selected guidance image as the guidewire is advanced along the path.

The method 1000 may, optionally, include a process 1014 of inserting the interventional instrument along the path. For example, the interventional tool 208 may be delivered over or along guidewire 820, 920 extending along the identified path. In some examples, when using the system 80 having the separable acoustic imaging device, the acoustic imaging device is removed from the working channel 206 before the interventional tool 208 is inserted over the guidewire 820. The guide channel 804 of the interventional tool 208 guides the interventional tool 208 over the guidewire 820. In other examples, when using the system 90 having the integrated acoustic imaging tool, the interventional tool 208 may be inserted through the working channel 206 over the guidewire 920 using the guide channel 904.

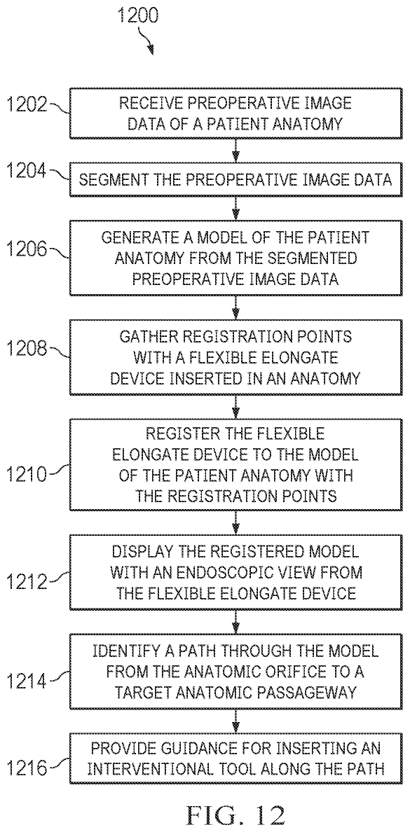

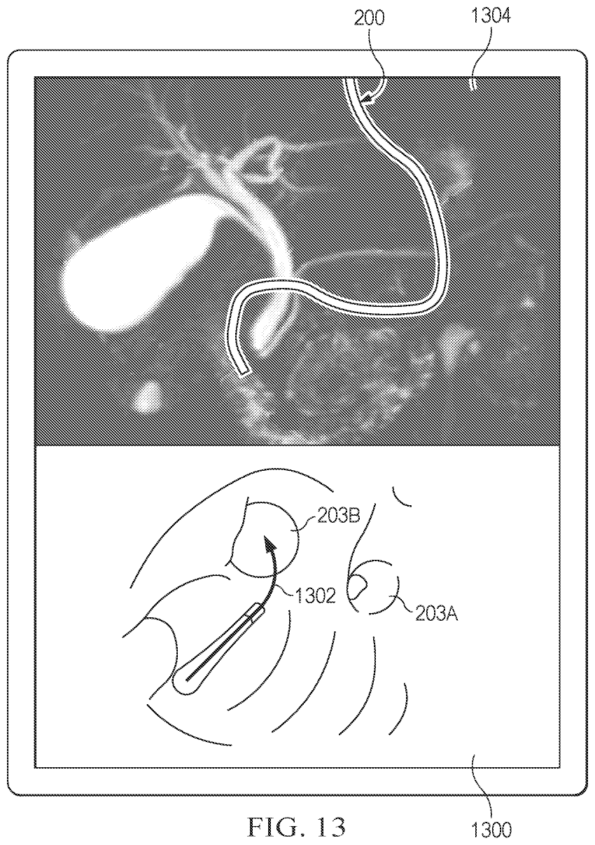

FIG. 12 is a flowchart illustrating a method 1200 for identifying and navigating hidden anatomic passageways. The method 1200 may use any of the principles described with respect to methods 300, 500, and 1000. The method 1200 incorporates pre-operative imaging. At a process 1202, preoperative image data of a patient anatomy is received. The pre-operative image data may be CT data or MRI data, among other types of data. In some examples, fiducial markers can be placed on or in the patient (e.g., voice box, esophageal sphincter, other rigid structures, etc.). The fiducial markers may assist in recognizing and identifying certain features in the pre-operative image data. At a process 1204, the preoperative image data is segmented. Rigid structures (e.g., esophageal sphincter, entrance to duodenum, papilla of vater, etc.) are identified in the preoperative image data. If fiducial markers were used, the fiducial markers may be identified in the preoperative image data as well. At a process 1206, a model of the patient anatomy is generated from the segmented preoperative image data, including the identified structures. At a process 1208, registration points may be gathered using a flexible elongate device (e.g., the flexible elongate device 200). Gathering the registration points may include touching the flexible elongate device 200 to the identified structures. The flexible elongate device 200 includes the localization sensor 402, so the location of the identified structures is determined. At a process 1210, the flexible elongate device 200 is registered to the model of the patient anatomy using the gathered registration points. At a process 1212, the registered model with an endoscopic view 1300 from the flexible elongate device 200 can be displayed, as shown in FIG. 13. In some examples, the display may include an image 1304 of the registered flexible elongate device 200 superimposed onto the segmented pre-operative image data. At a process 1214, a path (e.g., path 1302) through the model from the anatomic orifice 202 to a target in the hidden anatomic passageways 203A, 203B is identified. The path 1302 may be identified manually, or automatically using the control system 214. At a process 1216, guidance is provided for inserting the interventional tool 208 along the identified path. In some examples, the guidance is display of the path 1302 in the virtual endoscopic view 1300. In some examples, the guidance is the superimposition of the registered flexible elongate device 200 over the segmented pre-operative image data, as shown in image 1304. Although not illustrated, the image 1304 may also include the path 1302, for further guidance. At a process 1218, the interventional tool is delivered along the path using the path 1302 and the guidance.

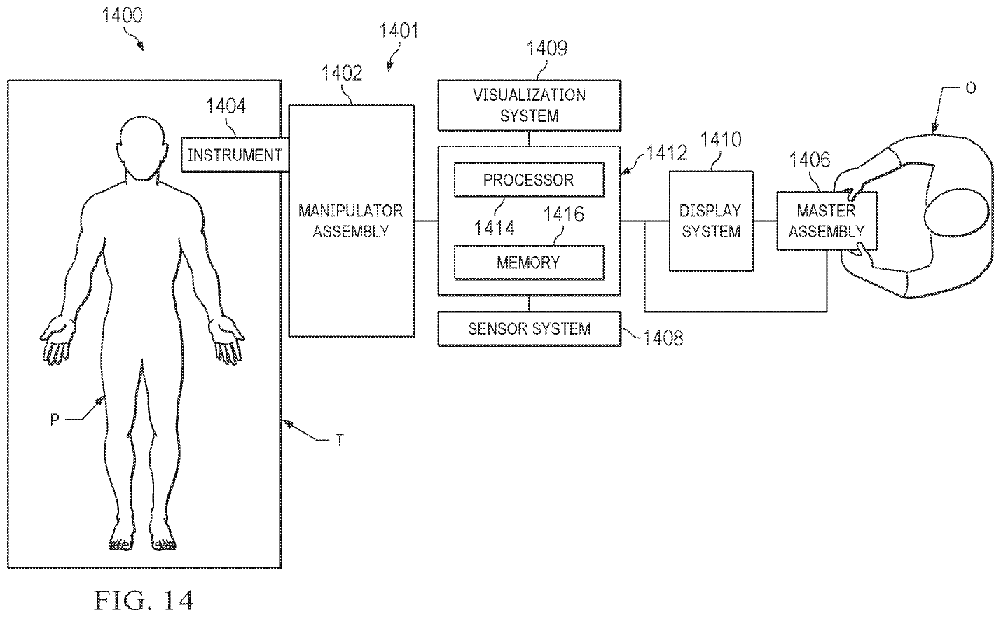

FIG. 14 illustrates a medical system 1400 that may include a manipulator assembly 1402 that controls the operation of a medical instrument 1404 such as a flexible elongate device (e.g., the flexible elongate device 200) in performing various procedures on a patient P. Medical instrument 1404 may extend into an internal site within the body of patient P via an opening in the body of patient P. The manipulator assembly 1402 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. The manipulator assembly 1402 may be mounted to and/or positioned near a patient table T. A master assembly 1406 allows an operator O (e.g., a surgeon, a clinician, a physician, or other user) to control the manipulator assembly 1402. In some examples, the master assembly 1406 allows the operator O to view the procedural site or other graphical or informational displays. In some examples, the manipulator assembly 1402 may be excluded from the medical system 1400 and the instrument 1404 may be controlled directly by the operator O. In some examples, the manipulator assembly 1402 may be manually controlled by the operator O. Direct operator control may include various handles and operator interfaces for hand-held operation of the instrument 1404.

The master assembly 1406 may be located at a surgeon's console which is in proximity to (e.g., in the same room as) a patient table T on which patient P is located, such as at the side of the patient table T. In some examples, the master assembly 1406 is remote from the patient table T, such as in in a different room or a different building from the patient table T. The master assembly 1406 may include one or more control devices for controlling the manipulator assembly 1402. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 1402 supports the medical instrument 1404 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 1412). The manipulator assembly 1402 may include a plurality of actuators (e.g., motors) that drive inputs on the medical instrument 1404 in response to commands, such as from the control system 1412. The actuators may include drive systems that move the medical instrument 1404 in various ways when coupled to the medical instrument 1404. For example, one or more actuators may advance medical instrument 1404 into a naturally or surgically created anatomic orifice. Actuators may control articulation of the medical instrument 1404, such as by moving the distal end (or any other portion) of medical instrument 1404 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 1404, such as for grasping tissue in the jaws of a biopsy device and/or the like or may be used to move or otherwise control tools (e.g., imaging tools, ablation tools, biopsy tools, electroporation tools, etc.) that are inserted within the medical instrument 1404.

The medical system 1400 may include a sensor system 1408 with one or more sub-systems for receiving information about the manipulator assembly 1402 and/or the medical instrument 1404. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 1404; a visualization system 1409 (e.g., using an optical imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 1404 or from some other location; and/or actuator position sensors such as resolvers, encoders, potentiometers, and the like that describe the rotation and/or orientation of the actuators controlling the medical instrument 1404.

The medical system 1400 may include a display system 1410 for displaying an image or representation of the procedural site and the medical instrument 1404. Display system 1410 and master assembly 1406 may be oriented so physician O can control medical instrument 1404 and master assembly 1406 with the perception of telepresence.

In some embodiments, the medical instrument 1404 may include a visualization system 1409, which may include an image capture assembly that records a concurrent or real-time image of a procedural site and provides the image to the operator O through one or more displays of display system 1410. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 1404. Additionally or alternatively, a separate endoscope, attached to a separate manipulator assembly, may be used with medical instrument 1404 to image the procedural site. The visualization system may be implemented as hardware, firmware, software or a combination thereof which interact with or are otherwise executed by one or more computer processors, such as of the control system 1412.

Display system 1410 may also display an image of the procedural site and medical instruments, which may be captured by the visualization system. In some examples, the medical system 1400 provides a perception of telepresence to the operator O. For example, images captured by an imaging device at a distal portion of the medical instrument 1404 may be presented by the display system 1410 to provide the perception of being at the distal portion of the medical instrument 1404 to the operator O. The input to the master assembly 1406 provided by the operator O may move the distal portion of the medical instrument 1404 in a manner that corresponds with the nature of the input (e.g., distal tip turns right when a trackball is rolled to the right) and results in corresponding change to the perspective of the images captured by the imaging device at the distal portion of the medical instrument 1404. As such, the perception of telepresence for the operator O is maintained as the medical instrument 1404 is moved using the master assembly 1406. The operator O can manipulate the medical instrument 1404 and hand controls of the master assembly 1406 as if viewing the workspace in substantially true presence, simulating the experience of an operator that is physically manipulating the medical instrument 1404 from within the patient anatomy.

In some examples, the display system 1410 may present virtual images of a procedural site that are created using image data recorded pre-operatively or intra-operatively, such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 1410 may display a virtual image that is generated based on tracking the location of medical instrument 1404. For example, the tracked location of the medical instrument 1404 may be registered (e.g., dynamically referenced) with the model generated using the pre-operative or intra-operative images, with different portions of the model correspond with different locations of the patient anatomy. As the medical instrument 1404 moves through the patient anatomy, the registration is used to determine portions of the model corresponding with the location and/or perspective of the medical instrument 1404 and virtual images are generated using the determined portions of the model. This may be done to present the operator O with virtual images of the internal procedural site from viewpoints of medical instrument 1404 that correspond with the tracked locations of the medical instrument 1404.

The medical system 1400 may also include the control system 1412, which may include processing circuitry that implements the some or all of the methods or functionality discussed herein. The control system 1412 may include at least one memory 1416 and at least one processor 1414 for controlling the operations of the manipulator assembly 1402, the medical instrument 1404, the master assembly 1406, the sensor system 1408, and/or the display system 1410. Control system 1412 may include instructions (e.g., a non-transitory machine-readable medium storing the instructions) that when executed by the at least one processor, configures the one or more processors to implement some or all of the methods or functionality discussed herein. While the control system 1412 is shown as a single block in FIG. 14, the control system 1412 may include two or more separate data processing circuits with one portion of the processing being performed at the manipulator assembly 1402, another portion of the processing being performed at the master assembly 1406, and/or the like. In some examples, the control system 1412 may include other types of processing circuitry, such as application-specific integrated circuits (ASICs) and/or field-programmable gate array (FPGAs). The control system 1412 may be implemented using hardware, firmware, software, or a combination thereof.

In some examples, the control system 1412 may receive feedback from the medical instrument 1404, such as force and/or torque feedback. Responsive to the feedback, the control system 1412 may transmit signals to the master assembly 1406. In some examples, the control system 1412 may transmit signals instructing one or more actuators of the manipulator assembly 1402 to move the medical instrument 1404. In some examples, the control system 1412 may transmit informational displays regarding the feedback to the display system 1410 for presentation or perform other types of actions based on the feedback.

The control system 1412 may include a virtual visualization system to provide navigation assistance to operator O when controlling the medical instrument 1404 during an image-guided medical procedure. Virtual navigation using the virtual visualization system may be based upon an acquired pre-operative or intra-operative dataset of anatomic passageways of the patient P. The control system 1412 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 1408 that is used to compute an (e.g., approximate) location of the medical instrument 1404 with respect to the anatomy of patient P. The sensor system 1408 may be used to register and display the medical instrument 1404 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 1408 may be used to compute the (e.g., approximate) location of the medical instrument 1404 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 1400 may further include operations and support systems (not shown) such as illumination systems, steering control systems, irrigation systems, and/or suction systems. In some embodiments, the medical system 1400 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

FIG. 15A is a simplified diagram of a medical instrument system 1500 according to some embodiments. The medical instrument system 1500 includes a flexible elongate device 1502 (e.g. flexible elongate device 200), a drive unit 1504, and a medical tool 1526 that collectively is an example of the medical instrument 1404 of the medical system 1400. The medical system 1400 may be a robot-assisted system, a non-robot-assisted system, or a hybrid robot-assisted and non-assisted system, as explained with reference to FIG. 14. A visualization system 1531, tracking system 1530, and navigation system 1532 are also shown in FIG. 15A and are example components of the control system 1412 of the medical system 1400. In some examples, the medical instrument system 1500 may be used for non-robot-assisted exploratory procedures or in procedures involving traditional manually operated medical instruments, such as endoscopy. The medical instrument system 1500 may be used to gather (e.g., measure) a set of data points corresponding to locations within anatomic passageways of a patient, such as patient P.

The elongate device 1502 is coupled to the drive unit 1504. The elongate device 1502 includes a lumen or channel 1521 through which the medical tool 1526 may be inserted. The elongate device 1502 navigates within patient anatomy to deliver the medical tool 1526 to a procedural site. The elongate device 1502 includes a flexible body 1516 having a proximal end 1517 and a distal end 1518. In some examples, the flexible body 1516 may have an approximately 3 mm outer diameter. Other flexible body outer diameters may be larger or smaller.

Medical instrument system 1500 may include the tracking system 1530 for determining the position, orientation, speed, velocity, pose, and/or shape of the flexible body 1516 at the distal end 1518 and/or of one or more segments 1524 along flexible body 1516, as will be described in further detail below. The tracking system 1530 may include one or more sensors and/or imaging devices. The flexible body 1516, such as the length between the distal end 1518 and the proximal end 1517, may include multiple segments 1524. The tracking system 1530 may be implemented using hardware, firmware, software, or a combination thereof. In some examples, the tracking system 1530 is part of control system 1512.

Tracking system 1530 may track the distal end 1518 and/or one or more of the segments 1524 of the flexible body 1516 using a shape sensor 1522 (e.g., a localization sensor). The shape sensor 1522 may include an optical fiber aligned with the flexible body 1516 (e.g., provided within an interior channel of the flexible body 1516 or mounted externally along the flexible body 1516). In some examples, the optical fiber may have a diameter of approximately 200 μm. In other examples, the diameter may be larger or smaller. The optical fiber of the shape sensor 1522 may form a fiber optic bend sensor for determining the shape of flexible body 1516. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 1516 may be determined using other techniques. For example, a history of the position and/or pose of the distal end 1518 of the flexible body 1516 can be used to reconstruct the shape of flexible body 1516 over an interval of time (e.g., as the flexible body 1516 is advanced or retracted within a patient anatomy). In some examples, the tracking system 1530 may alternatively and/or additionally track the distal end 1518 of the flexible body 1516 using a position sensor system 1520. Position sensor system 1520 may be a component of an EM sensor system with the position sensor system 1520 including one or more position sensors. Although the position sensor system 1520 is shown as being near the distal end 1518 of the flexible body 1516 to track the distal end 1518, the number and location of the position sensors of the position sensor system 1520 may vary to track different regions along the flexible body 1516. In one example, the position sensors include conductive coils that may be subjected to an externally generated electromagnetic field. Each coil of position sensor system 1520 may produce an induced electrical signal having characteristics that depend on the position and orientation of the coil relative to the externally generated electromagnetic field. The position sensor system 1520 may measure one or more position coordinates and/or one or more orientation angles associated with one or more portions of flexible body 1516. In some examples, the position sensor system 1520 may be configured and positioned to measure six degrees of freedom, e.g., three position coordinates X, Y, Z and three orientation angles indicating pitch, yaw, and roll of a base point. In some examples, the position sensor system 1520 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 1530 may alternately and/or additionally rely on a collection of pose, position, and/or orientation data stored for a point of an elongate device 1502 and/or medical tool 1526 captured during one or more cycles of alternating motion, such as breathing. This stored data may be used to develop shape information about the flexible body 1516. In some examples, a series of position sensors (not shown), such as EM sensors like the sensors in position sensor 1520 or some other type of position sensors may be positioned along the flexible body 1516 and used for shape sensing. In some examples, a history of data from one or more of these position sensors taken during a procedure may be used to represent the shape of elongate device 1502, particularly if an anatomic passageway is generally static.

FIG. 15B is a simplified diagram of the medical tool 1526 within the elongate device 15702 according to some embodiments. The flexible body 1516 of the elongate device 1502 may include the channel 1521 sized and shaped to receive the medical tool 1526. In some embodiments, the medical tool 1526 may be used for procedures such as diagnostics, imaging, surgery, biopsy, ablation, illumination, irrigation, suction, electroporation, etc. Medical tool 1526 can be deployed through channel 1521 of flexible body 1516 and operated at a procedural site within the anatomy. Medical tool 1526 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 1526 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 1526 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 1521 when the biopsy tool is within the channel 1521. The medical tool 1526 may be an image capture probe that includes a distal portion with a stereoscopic or monoscopic camera that may be placed at or near the distal end 1518 of flexible body 1516 for capturing images (e.g., still or video images). The captured images may be processed by the visualization system 1531 for display and/or provided to the tracking system 1530 to support tracking of the distal end 1518 of the flexible body 1516 and/or one or more of the segments 1524 of the flexible body 1516. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 1531. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 1516 of the elongate device 1502 to facilitate visual navigation of the elongate device 1502 to a procedural site and then is replaced within the flexible body 1516 with another type of medical tool 1526 that performs the procedure. In some examples, the image capture probe may be within the flexible body 1516 of the elongate device 1502 along with another type of medical tool 1526 to facilitate simultaneous image capture and tissue intervention, such as within the same channel 1521 or in separate channels. A medical tool 1526 may be advanced from the opening of the channel 1521 to perform the procedure (or some other functionality) and then retracted back into the channel 1521 when the procedure is complete. The medical tool 1526 may be removed from the proximal end 1517 of the flexible body 1516 or from another optional instrument port (not shown) along flexible body 1516.

In some examples, the elongate device 1502 may include integrated imaging capability rather than utilize a removable image capture probe. For example, the imaging device (or fiber-optic bundle) and the light emitters may be located at the distal end 1518 of the elongate device 1502. The flexible body 1515 may include one or more dedicated channels that carry the cable(s) and/or optical fiber(s) between the distal end 1518 and the visualization system 1531. Here, the medical instrument system 1500 can perform simultaneous imaging and tool operations.

In some examples, the medical tool 1526 is capable of controllable articulation. The medical tool 1526 may house control members or cables (which may also be referred to as pull wires), linkages, or other actuation controls (not shown) that extend between its proximal and distal ends to controllably bend the distal end of medical tool 1526, such as discussed herein for the flexible elongate device 1502. The medical tool 1526 may be coupled to a drive unit 1504 and the manipulator assembly 1402. In these examples, the elongate device 1502 may be excluded from the medical instrument system 1500 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sep. 30, 2008 and titled “Passive Preload and Capstan Drive for Surgical Instruments”), which are incorporated by reference herein in their entireties.

The flexible body 1516 of the elongate device 1502 may also or alternatively house cables, linkages, or other steering controls (not shown) that extend between the drive unit 1504 and the distal end 1518 to controllably bend the distal end 1518 as shown, for example, by broken dashed line depictions 1519 of the distal end 1518 in FIG. 15A. In some examples, at least four cables are used to provide independent up-down steering to control a pitch of the distal end 1518 and left-right steering to control a yaw of the distal end 1518. In these examples, the flexible elongate device 1502 may be a steerable catheter. Examples of steerable catheters, applicable in some embodiments, are described in detail in PCT Publication WO 2019/018736 (published Jan. 24, 2019 and titled “Flexible Elongated Device Systems and Methods”), which is incorporated by reference herein in its entirety.

In embodiments where the device 1502 and/or medical tool 1526 are actuated by a robot-assisted assembly (e.g., the manipulator assembly 1402), the drive unit 1504 may include drive inputs that removably couple to and receive power from drive elements, such as actuators, of the robot-assisted assembly. In some examples, the elongate device 1502 and/or medical tool 1526 may include gripping features, manual actuators, or other components for manually controlling the motion of the elongate device 1502 and/or medical tool 1526. The elongate device 1502 may be steerable or, alternatively, the elongate device 1502 may be non-steerable with no integrated mechanism for operator control of the bending of distal end 1518. In some examples, one or more channels 1521 (which may also be referred to as lumens), through which medical tools 1526 can be deployed and used at a target anatomical location, may be defined by the interior walls of the flexible body 1516 of the elongate device 1502.

In some examples, the medical instrument system 1500 (e.g., the elongate device 1502 or medical tool 1526) may include a flexible bronchial instrument, such as a bronchoscope or bronchial catheter, for use in examination, diagnosis, biopsy, and/or treatment of a lung. The medical instrument system 1500 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 1530 may be sent to the navigation system 1532, where the information may be combined with information from the visualization system 1531 and/or pre-operatively obtained models to provide the physician, clinician, surgeon, or other operator with real-time position information. In some examples, the real-time position information may be displayed on the display system 1410 for use in the control of the medical instrument system 1500. In some examples, the navigation system 1532 may utilize the position information as feedback for positioning medical instrument system 1500. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

The methods (e.g., 300, 500, 1000, 1200) described herein are illustrated as a set of operations or processes that may be performed in the same or in a different order than the order shown. One or more of the illustrated processes may be omitted in some examples of the method. Additionally, one or more processes that are not expressly illustrated in FIGS. 3, 5, 10, and 12 may be included before, after, in between, or as part of the illustrated processes. Further, processes of any of the methods may be used in another of the methods, even if not expressly stated. In some examples, one or more of the processes of the methods may be implemented, at least in part, by the control system (e.g., the control system 1412) executing code stored on non-transitory, tangible, machine-readable media that when run by one or more processors (e.g., the processors 1414 of the control system 1412) may cause the one or more processors to perform one or more of the processes.

One or more components of the embodiments discussed in this disclosure, such as control system 1412, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

In some instances well known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

While certain illustrative examples of the invention have been described and shown in the accompanying drawings, it is to be understood that such examples are merely illustrative of and not restrictive on the broad invention, and that the examples of the invention not be limited to the specific constructions and arrangements shown and described, since various other alternatives, modifications, and equivalents will be appreciated by those with ordinary skill in the art.

1. A system comprising:
a flexible elongate device including an optical imaging device and a working channel;
an acoustic imaging device carried by the flexible elongate device, the acoustic imaging device configured to generate a plurality of acoustic images of a region of interest;
an interventional tool extendable through the working channel of the flexible elongate device; and
a control system configured to:
receive the plurality of acoustic images of the region of interest from the acoustic imaging device and
provide guidance for inserting the interventional tool along a path through an anatomic orifice and an anatomic passageway of the region of interest, wherein the guidance is generated based on the plurality of acoustic images.

2. The system of claim 1, wherein providing the guidance includes:
generating a model of the region of interest from the plurality of acoustic images; and
displaying the model of the region of interest and a representation of the interventional tool advancing along the path through the anatomic orifice and the anatomic passageway of the region of interest.

3. The system of claim 2, wherein providing the guidance further includes:
displaying, with the model, a marker illustrating the path through the anatomic orifice and the anatomic passageway of the region of interest.

4. The system of claim 2, wherein the optical imaging device is configured to generate an image of a field of view and wherein providing the guidance further includes displaying a synthetic endoscopic image generated from the model, the synthetic endoscopic image corresponding to a perspective of the optical imaging device needed to align an interventional tool trajectory along the path.

5. The system of claim 4, wherein the control system is further configured to adjust a position and an orientation of the flexible elongate device until a real-time image of the field of view of the optical imaging device matches the synthetic endoscopic image.

6. The system of claim 2, wherein the control system is further configured to register the model to the flexible elongate device and the interventional tool.

7. The system of claim 1, wherein providing the guidance includes:
displaying the plurality of acoustic images of the region of interest, the plurality of acoustic images showing a guide device advancing along the path through the anatomic orifice and the anatomic passageway of the region of interest, wherein the interventional tool is configured to advance over the guide device.

8. The system of claim 7, wherein displaying the plurality of acoustic images includes displaying at least one selected guidance image of the plurality of acoustic images that includes the path.

9. The system of claim 8, wherein the control system is further configured to align a guide channel of the acoustic imaging device with the path visible in the selected guidance image.

10. The system of claim 9, wherein the guide device is a guidewire insertable through the guide channel aligned with the path through the anatomic orifice and the anatomic passageway.

11. The system of claim 1, wherein the optical imaging device is configured to generate an image of a field of view, wherein the region of interest is obscured by tissue surrounding the anatomic orifice while the plurality of acoustic images of the region of interest are received from the acoustic imaging device.

12. The system of claim 11, wherein the flexible elongate device includes an endoscope.

13. The system of claim 1, further comprising a localization device carried by the flexible elongate device and configured to provide the guidance for inserting the interventional tool.

14. The system of claim 13, wherein the localization device includes a localization sensor configured to reference the plurality of acoustic images to a common reference frame.

15. The system of claim 14, wherein the localization sensor is fixed relative to the acoustic imaging device.

16. The system of claim 14, wherein the localization sensor includes an optical fiber sensor.

17. The system of claim 13, wherein the localization device includes a guide device extendable from the flexible elongate device and visible in the plurality of acoustic images.

18. The system of claim 17, wherein the acoustic imaging device is integrated with the flexible elongate device and the guide device is a guidewire extendable through the working channel of the flexible elongate device.

19. The system of claim 17, further comprising an acoustic imaging tool including the acoustic imaging device, wherein the acoustic imaging tool is separable from the flexible elongate device and the guide device is a guidewire extendable through the acoustic imaging tool.

20. The system of claim 1, wherein the path has a substantially straight trajectory from the anatomic orifice, and wherein the interventional tool is generally rigid for traversing the path having the substantially straight trajectory.

21-72. (cancelled)
